(19) United States
(12) Patent Application Publication
(10) Pub. No.: US 2008/0286308 A1
(43) Pub. Date: Nov. 20, 2008

(54) Pathogenic Bovine Enterovirus, Vaccines, and Diagnostic Methods

(76) Inventors: Gary A. Anderson, Paolo, KS (US); Daniel J. Keil, Spring Hill, KS (US); Robert J. Flakus, Sioux Falls, SD (US)

Correspondence Address: Novartis Animal Health US Inc., 3200 Northline Avenue, Suite 300, Greensboro, NC, 27408, US

(21) Appl. No.: 10/581,184

(22) PCT Filed: Dec. 12, 2003
(86) PCT No.: PCT/EP04/14117

§ 371 Date: Jun. 29, 2006

Related U.S. Application Data
(60) Provisional application No. 60/529,349, filed on Dec. 12, 2003.

(51) Int. Cl. C12N 7/00; C12N 7/04; A61K 39/125; A61P 31/12; C12Q 1/70

(52) U.S. Cl. 424/216.1; 435/235.1; 435/236; 435/5

(57) ABSTRACT

The isolation of a novel pathogenic bovine enterovirus is described. The novel pathogenic bovine enterovirus is used to develop antibodies and immunogenic compositions against the novel pathogenic bovine enterovirus. Diagnostic assays are described.

### BACKGROUND OF INVENTION

This invention relates to the isolation of an unique pathogenic bovine enterovirus, a vaccine against the virus, antibodies that bind to the virus, and diagnostic methods thereof. Bovine enterovirus is a picornovirus. It has a single stranded sense RNA and is approximately 25-30 nm in diameter. The viron is a non-enveloped icosahedral. The virus replicates in the cytoplasm of the infected cell. Bovine enterovirus is insensitive to ether. Bovine enteroviruses have been well characterized. The nucleotide sequence of an bovine enterovirus was completed in 1988 (Earle J A, et al., “The complete nucleotide sequence of a bovine enterovirus”, J. Gen. Virol. 69:Part 2, pp. 253-63 (February 1988)). ELISA assays have been developed to identify bovine enteroviruses (Hofner M C, “An indirect sandwich ELISA for the identification of bovine enteroviruses”, J. Virol. Methods, 41:2 pp. 239-43 (February 1993)) and for detecting antibodies in animals that bind to bovine enteroviruses (Zhang A Q, et al., “A capture antibody ELISA for detection of antibodies against bovine enterovirus”, J. Virol. Methods 24:1-2, pp 223-6 (April-May 1989)). Neutralizing antibodies can be produced using synthetic peptides (Smyth M S, et al., “Characterization of neutralizing antibodies to bovine enterovirus elicited by synthetic peptides”, Arch. Virol. 126:1-4, pp 21-33 (1992)). While bovine enteroviruses have had some cross-antigenicity with foot-and-mouth disease virus (Andersen A A, “Cross reaction between bovine enterovirus and South African Territories 15 foot-and-mouth disease virus”, Am. J. Vet. Res. 39:1, pp 59-63 (January 1978)), it is generally well accepted that bovine enteroviruses are safe in both bovine and humans. In fact, others have suggested using bovine enteroviruses for cancer therapy (see for example, Taylor M W, et al., “Viruses as an aid to cancer therapy: Regression of solid and ascites tumors in rodents after treatment with bovine enteroviruses”, PNAS 68:4, pp. 836-40 (April 1971) and Smyth M, et al., “Bovine enteroviruses as an oncolytic virus: Foetal calf serum facilitates its infection of human cells”, Int. J. Mol. Med., 10:1, pp. 49-53 (July 2002)). Recently, examination of tissue samples from bovine having a respiratory disease reveal that bovine enterovirus is the causative agent of the disease. This particular bovine enterovirus can be isolated and be demonstrated to cause respiratory disease in cattle. As such, a vaccine to the bovine enterovirus is useful for the prevention of disease in cattle. Antibodies to this particular bovine enterovirus is useful to have because antibodies can be used for diagnostic assays.

### BRIEF DESCRIPTION OF THE INVENTION

It is an object of this invention to isolate and characterize a novel pathogenic bovine enterovirus. It is a further object of this invention to have assays for diagnosing this virus in animals and methods for performing the assays. It is yet another object of this invention to have an immunogenic composition to this novel pathogenic bovine enterovirus, methods for producing this immunogenic composition, and methods for stimulating an immune response in animals using this immunogenic composition.

It is an object of this invention to have polyclonal antibodies and monoclonal antibodies which bind to this novel pathogenic bovine enterovirus. It is another object of this invention to use these polyclonal antibodies and/or monoclonal antibodies for a diagnostic assay to test animals for infection with this novel pathogenic bovine enterovirus. It is another object of this invention to use these polyclonal antibodies and/or monoclonal antibodies during the production of an immunogenic composition.
It is an object of this invention to have an immunogenic composition which generates an immune response in an animal to pathogenic bovine enterovirus. It is a further object of this invention that the immunogenic composition contain inactivated pathogenic bovine enterovirus, attenuated pathogenic bovine enterovirus, antigenic proteins or polypeptides from pathogenic bovine enterovirus, nucleotide sequences encoding antigenic proteins or polypeptides, and/or other immune stimulatory compositions which, when administered to an animal, causes the animal to generate an immune response to pathogenic bovine enterovirus.

### DETAILED DESCRIPTION OF THE INVENTION

This invention concerns the isolation of a novel pathogenic bovine enterovirus. This invention also concerns the development of diagnostic assays for this novel pathogenic bovine enterovirus and an immunogenic composition for treating cattle with this disease and/or preventing cattle from becoming sick with this disease.

An immunogenic composition against this pathogenic bovine enterovirus can contain either inactivated pathogenic bovine enterovirus or attenuated pathogenic bovine enterovirus derived from an inoculum processed from infected bovine lung tissue or other bovine tissue exhibiting the characteristic lesions of this particular pathogenic bovine enterovirus or from deep nasal swabs. Functional derivatives of the pathogenic bovine enterovirus including subunit, vector, recombinant, and synthetic peptide immunogenic compositions or the like are also part of this invention.

### Isolation of Pathogenic Bovine Enterovirus

Pathogenic bovine enterovirus can be isolated by taking deep nasal swabs using long Dacron-tipped swabs from bovine with severe nasal discharge which can optionally contain blood. The infected animal also exhibits an increased body temperature, at approximately 103-104° F., decreased feeding (also called “off feeding”), and a depressed attitude. Infected bovines have a low incidence of animal mortality. The swabs can be stored with the tips in 2 ml minimum essential medium (MEM) containing 1% fetal bovine serum (FBS) and gentamicin (100 μg/ml) at −20° C. until ready for use. The swabs can also be stored in any other pharmaceutically acceptable aqueous solution (such as physiological saline, Ringers solution, Hank's Balanced Salt Solution, and the like). Forty-three samples are obtained and used to isolate the pathogenic entity.

Each deep nasal swab sample is thawed at room temperature then passed through a 0.2 μsyringe filter (Pall Gelman, Ann Arbor, Mich.) to obtain the filtered sample. Each filtered sample is then used to inoculate three different eukaryotic cell monolayers in 96-well plates. Each well contains one of the following cell lines at approximately 80%-100% confluence: Madin-Darby bovine kidney (MDBK), Rhesus monkey kidney (RMK), and human rectal tumor cells (HRT) cell lines. 20 μl of each filtered sample is added to one well, with a total of four wells being inoculated for each filtered sample. Each well contains 100 μl MEM and gentamicin (100 μg/ml). The plates are incubated for 30-60 minutes at 35-37° C. in 4% CO₂ atmosphere. Next, 100 μl of MEM and gentamicin (100 μg/ml) are added to each well and the plates are incubated for 3-5 days at 35-37° C. in 4% CO₂ atmosphere.
After the second incubation (for 3-5 days), the cells are examined via light microscopy for cytopathic effects, such as the cells rounding up, becoming pyknotic, and releasing from the bottom surface of the well. Cell death occurs. If no cell cytopathic effects are observed, the cells are scraped off the bottom of the plate well, isolated, and frozen at −70° C. for approximately 30 minutes or longer and then thawed. Then 20 μl of the freeze-thaw sample is added to another well containing the same cell line as previously used at approximately 80%-100% confluence and processed as described above. Again, if no cytopathic effects are observed, the sample is again freeze-thawed and reincubation of another well of the same cell line occurs. This freeze-thaw process occurs a maximum of three times for all samples that lacked obvious cytopathic effects (CPE).
The results are listed below:
28 samples show aggressive, high levels of CPE on MDBK cells
7 of those 28 show cytopathic changes that were considered to be +/− on HRT cells
no other sample shows signs of CPE on HRT cells or MDBK cells
no CPE is observed in any of the samples after 3 passages on RMK cells
   Pathogenic bovine enterovirus can also be isolated as an inoculate from lung tissue or naso-sinus tissue from bovines which exhibit severe nasal discharge which can optionally contain blood. The infected animal also exhibits an increased body temperature, at approximately 103-104° F., decreased feeding (also called “off feeding”), a depressed attitude, and low incidence of animal mortality. Such bovine are destroyed and their lung tissue removed. The lung tissue is examined using light microscopy. Histopatholgical evaluation indicates bronchitis and/or bronchiolitis with lesions showing mononuclear leukocyte infiltration and degenerating cells; alveolar septae may be thickened with cellular infiltrates; and excess debris may be present in alveoli. These characteristics, along with the above indicated physical examination, indicate the presence of the pathogenic bovine enterovirus.
   The desired tissue is homogenized with a pharmaceutically acceptable aqueous solution such that the tissue homogenate comprises ten percent weight/volume amount of the homogenate. The homogenate is then passed through filters with pore diameters ranging between approximately 0.1 to 10 micron, preferably through a series of filters having decreasing pore size (such as 1 micron, 0.6 micron, and 0.2 micron) to produce a filtered sample containing pathogenic bovine enterovirus. It may be preferable that the filtered sample contain biological particles in size no larger than about 1 micron, more preferably no larger than 0.2 microns in size (enterovirus have a particle size of 0.23 to 0.28 microns in diameter and should not pass through a 0.2 micron filter).
   To produce a purified form of pathogenic bovine enterovirus, the filtered sample can be inoculated into a series of in-vitro cell preparations as described above. Alternatively, cell preparations with mammalian organ cells such as kidney, liver, heart and brain, lung, spleen testicle, turbinate, white and red blood cells and lymph node, as well as insect and avian embryo preparations can be used. Culture media suitable for these cells preparations include those supporting mammalian cell growth such as fetal calf serum and agar, blood infusion agar, brain-heart infusion glucose broth and agar and the like. Preferably the mammalian cells are MDBK cells.

After inoculating the cell preparation with the filtered sample and growing the culture, individual clumps of cultured cells are harvested, freeze-thawed, and reintroduced into sterile culture medium with cells. The culture fluid from the final culture of the series provides the purified form of the pathogenic bovine enterovirus. Also, after a series of repeated harvests have been made, the culture can be grown, the culture fluid collected, and the fluid used as an inoculum for a culture of different cellular species. In this fashion, the pathogenic bovine enterovirus can be attenuated such that the culture fluid from the differing species culture provides the purified form of the pathogenic bovine enterovirus.

### Characterization of Pathogenic Bovine Enterovirus

The pathogenic bovine enterovirus may be characterized by determining physiochemical properties (such as size, sensitivity to lipid solvents, sensitivity to proteases, binding to lectins, etc.), by determining the DNA sequences and/or amino acid sequences, etc. Below are the protocols and results of several assays to identify a virus as a bovine enterovirus.

### Nucleic Acid Determination

Halogenated nucleosides (5-bromo-2-deoxyuridine, 5-fluor-2-deoxyuridine and 5-iodo-2-deoxyuridine) are 3 different chemically related metabolic inhibitors which are often used to identify the type of nucleic acid contained within a specific animal virus. These halogenated nucleosides as well as mitomycin c inhibit the replication of DNA viruses and have no effect on the replication of most RNA viruses. These nucleosides and mitomycin c exert an inhibitory effect on Retroviridae because of the fact that early DNA synthesis is required for these viruses. The effect of these chemical agents is reversible by thymidine.

Grow a confluent layer of MDBK cells in eighteen T-25 cm² cell culture flasks in Dulbecco's Modified Eagle's Medium (DME). Upon reaching confluence, one decants the DME and replaces it with DME deficient in thymidine containing 100 μg/ml of 5-iodo-2-deoxyuridine (IDUR). After incubation at 37° C. for 24 hours, remove the thymidine deficit, IDUR⁺ DME from the cell cultures. Inoculate four MDBK cell cultures with each of the three control viruses (IBRV (infectious bovine rhinotracheitis virus), a known bovine enterovirus (BEV type 3 strain PS89, ATCC deposit VR-755), and bovine viral diarrhea virus (BVDV type 1a strain KY-22)) and the isolated virus suspected of being a pathogenic bovine enterovirus for a total of 16 infected cell cultures. Incubate the infected cell cultures for 1 hour at 37° C. and remove the unadsorbed virus by washing each flask with 10 ml of phosphate buffer saline (PBS). Add 10 ml of DME growth media with 20 μg/ml of thymidine to two of the four infected cell cultures for each of the respective test viruses and add 10 ml of DME growth media containing 100 μg/ml IDUR to the half of the infected test virus cultures. Include another two T-25 cm² MDBK culture flasks infected with each of the four respective viruses as untreated control flasks. Incubate the cultures at 37° C. and examine daily for CPE. When CPE is visible in the virus infected flasks containing DME with thymidine (the amount of time is dependent on the particular virus in question), harvest all sets of the test cultures infected with the same virus as well as the control cultures for that virus, titrate for viral infectivity and compare. The same protocol can be used for mitomycin c by replacing the IDUR with 20 μg/ml mitomycin c.
The presence of IDUR or mitomycin C has a negative effect on the infectivity of IBRV, a DNA virus. This negative effect is mitigated by the addition of thymidine to the cell culture. The presence of IDUR or mitomycin C has no effect on the infectivity of BEV Type 3 and BVDV which are both RNA viruses. The addition of thymidine to the cell culture has no impact. For the virus isolated as above and suspected of being a pathogenic bovine enterovirus, the presence of IDUR or mitomycin C has no effect on its infectivity and thymidine added to the cell culture also causes no change. This assay can be used to confirm that the virus suspected of being a pathogenic bovine virus is an RNA virus.

### Ether Sensitivity

Organic solvents remove essential lipids from the nucleocapsid or outer envelope of viruses containing such material in their structure and this treatment thereby renders the agent non-infective. Therefore, most animal viruses which possess an envelope surrounding their nucleocapsids are sensitive to lipid solvents such as ether.

Using the suspected pathogenic bovine enterovirus and the four control viruses (BAV (bovine adenovirus type 5, bartha strain), BEV (bovine enterovirus type 3 strain PS89, ATCC deposit VR-755), BVDV (bovine virus diarrhea virus, type 1a, strain KY-22), and IBRV (infectious bovine rhinotracheitis virus)), mix four parts of each test viral suspension to one part ether and incubate mixture at 4° C. for 24 hours on tube roller. Incubate a negative control of non-ether treated viral suspension concurrently under same incubation conditions. After 24 hours, centrifuge all treatment tubes at 1000×g for 20 minutes. Titrate the aqueous layer from ether treatments and non-treated control suspensions for viral infectivity in MDBK cell line. A decrease in viral titer of 1.0 log₁₀ or more in the ether treated samples as compared to the non-treated virus suspension indicates susceptibility to diethyl ether. For both BAV Type 5 and BEV Type 3, ether treatment has no impact on the viruses' infectivity, indicating that these viruses are non-enveloped viruses. For IBRV and BVDV, the ether treatment reduced the viruses' infectivity by at least 1.0 log₁₀, indicating that these viruses are enveloped viruses. For the virus isolated above and suspected of being a pathogenic bovine enterovirus, the ether treatment has no affect on the virus' infectivity, indicating that this virus is a non-enveloped virus, similar to BEV Type 3.

### pH Liability/Stability

Exposure of viruses to pH 3 for 30 minutes reduces the infectivity of some viruses (for example, rhinoviruses which are members of the Picornaviridae family) while causing no effect on other viruses such as enteroviruses which are also members of the same family. Rhinoviruses become susceptible to acidic conditions and lose activity below pH 5. Viruses can be characterized as acid labile (loss of 1.0 log₁₀ or more) or acid stabile (no loss in titer or loss of less than 1.0 log₁₀).

Divide a viral suspension of the isolated virus suspected of being a pathogenic bovine enterovirus, and four control viruses (BEV (bovine enterovirus type 3, strain PS89, ATCC deposit VR-755), IBRV (infectious bovine rhinotracheitis virus), BRSV (Bovine Respiratory Syncytial Virus, Lehmkuhl 375 Strain), and bovine rhinovirus type 2, strain EC-11 (ATCC deposit VR-392)) into four equal parts. Adjust the pH of DME so that there is DME at pH 3, pH 5, and pH 7.2. Mix one part of each respective viral suspension with nine parts of DME growth media at each test pH. Allow the mixtures to incubate at 25° C. for two hours. Titrate all samples for viral infectivity in MDBK cell line and compare.
For IBRV, BRSV, and bovine rhinovirus type 2, each viruses' infectivity is reduced by at least 1.0 log₁₀ at pH 5 and pH 3. In contrast, BEV type 3 and the virus suspected of being a pathogenic bovine enterovirus, the infectivity rate are constant across the pH ranges. This assay helps confirm that the isolated pathogenic virus is a bovine enterovirus virus.

### Cationic Stabilization

High concentrations of divalent cations, such as magnesium chloride, stabilize certain viruses (enteroviruses and reoviruses) when they are subjected to 50° C. for 1 hour, while they increase thermo-inactivation of other viruses (adenoviruses, herpes simplex, and vaccinia).

Using the isolated virus suspected of being a pathogenic bovine enterovirus and four control viruses (BEV (bovine enterovirus type 3, strain PS89, ATCC deposit VR-755), IBRV (infectious bovine rhinotracheitis virus), BAV (bovine adenovirus type 5, bartha strain), and a bovine rotavirus (NCDV-Lincoln strain)), dilute each of the respective virus isolates ten fold in 1 M MgCl₂ and in distilled water (control). Incubate the tubes in a water bath at 50° C. for 1 hour. After incubation, titrate all paired treatment samples for infectivity in MDBK cell line and MA-104 cell line (depending on the virus) and compare to non-heated control viral infectivity. For IBRV and BAV type 5, the viruses' infectivity rate falls to zero after being heated to 50° C. for 1 hour in water. The addition of 1 M MgCl₂ to the viral suspension does not improve the results for IBRV and BAV type 5. For BEV type 3 and IBRV, the viruses' infectivity falls to zero after being heated to 50° C. for 1 hour in water. The addition of 1 M MgCl₂ to the viruses' suspensions improves the results with the viruses' infectivity decreasing by approximately 1.0 log₁₀ compared to the non-heated controls. For the isolated virus suspected of being a pathogenic bovine enterovirus, the addition of 1 M MgCl₂ to the viral suspension allows the virus' infectivity to decrease by approximately 1.0 log₁₀ compared to the non-heated control. This assay helps confirm that the isolated, pathogenic virus is a bovine enterovirus.

### Confirmation of Isolation of Pathogenic Bovine Enterovirus

Pathogenic bovine enterovirus obtained from infected bovine, as described above, can be used to inoculate healthy bovine in order to help determine the physiochemical properties of the virus and to help assess treatment modalities and pathogenicity of the virus.

Healthy, new born calves are obtained and kept in a pathogen-free environment using the protocol described in Bjorneby, J. M., et al., Monoclonal antibody immunotherapy in nude mice persistently infected with Cryptosporidium parvum, Infect. Immun. 59, pp. 1172-1176 (1991). 5 ml of the filtered sample obtained as described above is used to inoculate the calves via a spraying the inoculum into the nostrils of the calves with a syringe. Inoculated calves develop clinical signs and symptoms of infected bovine. Lungs, liver, kidney, spleen, heart and brain from these inoculated calves are harvested eight to fourteen days after inoculation and examined for evidence of infection.

A sample of the pathogenic bovine enterovirus (identified as Strain 3A115, NAH-1013) was deposited with American Type Culture Collection (ATCC), 10801 University Blvd., Manassas, Va., 20110-2209 on Nov. 17, 2004 under the protocols of the Budapest Treaty and assigned ATCC accession number H-33555A.

### Immunogenic Compositions

Any filtered sample (from deep nasal swabs or tissue homogenates) can be used to produce an immunogenic composition. The inventors arbitrarily choose one filtered sample from deep nasal swab for production of an immunogenic composition. Seed virus, determined to be pure and free of adventitious agents (using standard culturing methods to screen for the presence of bacteria and other viruses), are inoculated (0.001-0.00001 MOI (multiplicity of infection)) onto MDBK cell monolayers two to three days after the MDBK cells are planted. One can use 75 cm², 150 cm² or any other size flask. While there is no upper size limitation, it may be preferable to use no flasks no larger than 1750 cm². The MDBK cells are grown in MEM and gentamicin (100 μg/ml) at 35-37° C. in 4% CO₂ atmosphere. Complete cytopathic effects develop 12-72 hours post-inoculation (typically 24 hours), and the viral fluids containing the virus are harvested by pooling into sterile containers which can range in size between 1 liter and 50 liters (most commonly 20 liters). The viral fluids can optionally be stored between 4-8° C. prior to inactivation. The viral fluids are chemically treated with beta-proriolactone (BPL), 1 ml per liter of viral fluids with constant stirring, to inactivate the virus. Inactivation typically takes 24-36 hours at 22-27° C. with constant pH adjustment using 5N NaOH to maintain pH 7.2-7.4. Alternative inactivating agents that can be used include, but are not limited to, binary ethylamine (BEI), aldehyde regents including formalin, formaldehyde, acetaldehyde and the like; reactive acidic alcohols including cresol, phenol and the like; acids such as benzoic acid, benzene sulfonic acid and the like; lactones such as beta proiolactone and caprolactone; activating lactams, carbodiimides and carbonyl diheteroaromatic compounds such as carbonyl diimidazole; and saponins. Irradiation such as with ultraviolet and gamma irradiation and/or heat can also be used to inactivate or kill pathogenic bovine enterovirus. After inactivation, the inactivated viral fluids are optionally mixed with an adjuvant such that the final concentration of the adjuvant is approximately 18-24% final dose volume. Adjuvants can include, but are not limited to, aluminum salts, such as aluminum hydroxide and aluminum phosphate; polymers, such as POLYGEN®, DEAE dextran, dextran sulfate, and methylacrylates; dimethylodecylammonium bromide; poxvirus proteins, such as BAYPAMUNE®, AVIRDINE®, lipid A; oils (and oil and water mixtures), such as EMULSIGEN®, EMULSIGEN PLUS®, SUPRIMM®; animal oils, such as squalane and squalene; mineral oils, such as Drakeol and Montanides; vegetable oils, such as peanut oil; block co-polymers; triterpenoid glycosides, such as saponin, Quil A, and QS21; detergents, such as Tween-80 and Pluronic; bacterial component adjuvants, such as Freund's incomplete adjuvant, Corynebacterium, Propionibacterium, and Mycobacterium; interleukens, monokines, and interferons; liposomes; ISCOMs; synthetic glycopeptides, such as muryamyl dipeptides and derivatives thereof, cholera toxin; or combinations thereof. Preferably, the adjuvant is an oil and water adjuvant such as SUPRIMM® (Novartis Animal Vaccine, Inc., Bucyrus, Kans.). A pharmacologically acceptable carrier such as but not limited to, saline solution, DEAE dextran, lactose, and polypropylene glycol, can be added to the immunogenic composition.

Alternatively, pathogenic bovine enterovirus can be attenuated by its repeated passage or growth in culture with non-bovine mammalian cells or avian cells so that the ability of the pathogenic bovine enterovirus to virulently reproduce is lost. The attenuated virus can be optionally mixed with the various adjuvants and pharmaceutically acceptable carriers mentioned above prior to administration to the animal.
Antigen (viral) quantity per dose for an inactivated viral immunogenic composition may range between 10⁴ and 10¹² TCID₅₀/dose, preferably between 10⁴ and 10⁸ TCID₅₀/dose, and more preferably approximately 10⁵ TCID₅₀/dose. The titration is accomplished by measurements using antibodies that bind to pathogenic bovine enterovirus in an immunoassay such as ELISA, RIA, IFA, or by enzyme substrate detection test. The dose volume of an immunogenic composition may range between 1 ml and 5 ml, with 2 ml being preferred. This invention's immunogenic composition can prevent and treat pathogenic bovine enterovirus infections in bovine. For effective prophylactic and anti-infectious use in-vivo, the immunogenic composition contains killed or attenuated pathogenic bovine enterovirus, immunogenic viral components, anti-idiotypic antibodies, or other known immunogenic compositions. The immunogenic composition may be administered alone, in combination with a pharmaceutical carrier that is compatible with bovine, in combination with an adjuvant, or in combination with a pharmaceutical carrier and an adjuvant. The immunogenic composition may be administered orally, parenterally, intranasally, intravenously, intramuscularly, intravaginally, intraanally, or any other known route of administration. Factors bearing on the dosage of an immunogenic composition include, for example, the age, weight, and level of maternal antibody in the bovine. The range of a given dose is approximately between 10⁴ and 10¹² TCID₅₀/dose, preferably between 10⁴ and 10⁸ TCID₅₀/dose, and more preferably approximately 10⁵ TCID₅₀/dose, in 1 ml to 5 ml doses. Protective immunity may be achieved with one dose or multiple doses may be administered two, three, or more weeks apart to provide protective immunity to pathogenic bovine enterovirus.
The immunogenic composition for pathogenic bovine enterovirus can be administered in a variety of different dosage forms. An aqueous medium containing the killed or attenuated pathogenic bovine enterovirus may be desiccated and combined with pharmaceutically acceptable inert excipients and buffering agents, such as lactose, starch, calcium carbonate, or sodium citrate, when formed into tablets, capsules, and the like. These combinations may also be formed into a powder or suspended in an aqueous solution such that these powders and/or solutions can be added to animal feed or to the animals' drinking water. These powders or solutions can be suitably sweetened or flavored by various known agents to promote uptake of the immunogenic composition orally by the bovine.
For parenteral administration, the immunogenic composition can be combined with pharmaceutically acceptable carriers such as saline solution, water, propylene glycol, and the like. The immunogenic composition may also include emulsifying and/or suspending agents as well, together with pharmaceutically acceptable diluent to control the delivery and the dose amount of the immunogenic composition.
Besides killed and attenuated pathogenic bovine enterovirus immunogenic compositions, one can use a subunit immunogenic composition or other type of immunogenic composition which presents to the animal the antigenic components of pathogenic bovine enterovirus. The antigenic component is a protein, glycoprotein, lipid-conjugated protein or glycoprotein, a modified lipid moiety, or other viral component which, when injected into an animal, stimulates an immune response in the animal such that the animal develops protective immunity against wild-type pathogenic bovine enterovirus. For a subunit immunogenic composition, the pathogenic bovine enterovirus can be cultured on animal cells, as described above. The cell culture can be homogenized and an immunogenic composition can be isolated by passage of the cell culture homogenate over the appropriate column or through the appropriate pore size filter or via centrifugation of the cell culture homogenate. One may preferably screen the immunogenic composition for the presence of virulent pathogenic bovine enterovirus and discard the immunogenic composition should virulent pathogenic bovine enterovirus exist.

If the antigenic component is a protein, then one can isolate the nucleic acid which encodes that protein and generate a immunogenic composition that contains that isolated nucleic acid. The nucleic acid encoding the antigenic component can be placed on a plasmid downstream of a signal sequence of an eukaryotic promoter. That plasmid can contain one or more selectable markers and be transfected into an attenuated prokaryotic organism, such as Salmonella spp., Shigella spp., or other suitable bacteria. The bacteria is then administered to the bovine so that the bovine can generate a protective immune response to the antigenic component. See, for example, U.S. Pat. No. 5,887,159 to Hone, et al. and U.S. Patent Application Publication Number 2002-0193330 by Hone, et al. Alternatively, the nucleic acid encoding the antigenic component can be placed downstream of a prokaryotic promoter, have one or more selectable markers, and be transfected into an attenuated prokaryotic organism such as Salmonella spp., Shigella spp., or other suitable bacteria. The bacteria is then administered to the eukaryotic subject for which immune response to the antigen of interest is desired. See, for example, U.S. Pat. No. 6,500,419 to Hone, et al. Additionally, a nucleic acid encoding a proteinous antigenic component of pathogenic bovine enterovirus can be administered to the mucosal tissue of a bovine to generate an immunogenic response. See, for example, U.S. Pat. No. 6,110,898 to Malone et al. Alternatively, a naked polynucleotide encoding an antigenic component of pathogenic bovine enterovirus can be electroporated into a bovine to generate an immune response using the methods similar to those described within Drabick, J. J.; et al.; Cutaneous transfection and immune responses to intradermal nucleic acid vaccination are significantly enhanced by in vivo electropermeabilization, Mol. Ther. Vol 3(2); pp. 249-55 (2001).

For a subunit immunogenic composition, the nucleic acid encoding a proteinous antigenic component of pathogenic bovine enterovirus can be cloned into a plasmid such as those described in International Patent Application Publication Number WO 00/32047 (Galen) and International Patent Application Publication Number WO 02/083890 (Galen). Then the plasmid can be transfected into bacteria and the bacteria can produce the desired antigenic protein. One can isolate and purify the desired antigenic protein by a variety of methods described in both patent applications.

### Antibodies

Polyclonal antibodies can be produced through use of pathogenic bovine enterovirus as an antigenic substance to raise an immune response in animals. The culture fluid or filtered sample or purified viruses prepared as described above can be administered with or without a stimulating adjuvant to a non-bovine animal such as a horse, goat, mouse, rabbit, fish, or chicken. Rabbits are used for polyclonal antibodies for this invention. After repeated immunization, portions of blood serum can be removed and antigenically purified using immobilized antibodies to those disease specific antibodies typically found in the serum of the bled animal. Further treatment of the semi-purified serum by chromatography on, for example, a saccharide gel column with physiological saline and collection of the proteinaceous components of molecular weight at least 10 kDa provides purified polyclonal antibodies for use in treatment or diagnosis.

### Monoclonal Antibody Preparation

Monoclonal antibodies can be produced by the hybridoma technique. After immunization of a mouse, pig, rat, rabbit or other appropriate animal with pathogenic bovine enterovirus (purified as described above) or filtered sample (described above) or cell culture supernatant (described above), the spleen of the animal can be removed and converted into a whole cell preparation. Following the method of Kohler and Milstein (Kohler et al., Nature, 256, 495-97 (1975)), the immune cells from the spleen cell preparation can be fused with myeloma cells to produce hybridomas. Culturing of the hybridomas and testing the culture fluid against fluid or inoculum containing pathogenic bovine enterovirus allows isolation of hybridomas that produce monoclonal antibodies to pathogenic bovine enterovirus. Introduction of the hybridoma into the peritoneum of the host species will produce a peritoneal growth of the hybridoma. Collection of the ascites fluid yield body fluid containing the monoclonal antibody to pathogenic bovine enterovirus. Also cell culture supernatant from the hybridoma cell lines can be used to isolate monoclonal antibodies. Preferably the monoclonal antibodies are produced by a murine derived hybrid cell line wherein the antibody is an IgG or IgM type immunoglobulin. Monoclonal antibodies can be used for various diagnostic and therapeutic compositions and methods, including passive immunization and anti-idiotype antibodies for immunogenic composition preparations.

Eight weeks before the date of hybridoma fusion, inoculate BALB/c AnN mice i.p. with a 1:1 suspension of heat inactivated pathogenic bovine enterovirus and complete Freund's adjuvant (CFA). The amount of BPL inactivated pathogenic bovine enterovirus used depends on the immunogenicity and toxicity of the inactivated virus. Use a maximum of 0.3 ml CFA per mouse. Five weeks after the initial immunization, inject mice with a booster dose of BPL inactivated pathogenic bovine enterovirus and CFA. One week prior to the fusion, immunize the mice i.p. with BPL inactivated pathogenic bovine enterovirus in saline. Two days before the fusion, immunize the mice i.p. with BPL inactivated pathogenic bovine enterovirus and saline. Two days before the fusion, inoculate the mice i.v. with BPL inactivated pathogenic bovine enterovirus and saline.
Euthanize a mouse that received injections of BPL inactivated pathogenic bovine enterovirus, remove the spleen by aseptic procedures, and transfer the spleen to a sterile Petri dish containing 5 ml cold serum free Dulbecco's Modified Eagle Media (DMEM). Use a scalpel blade to slit the spleen along the long axis and gently scrape along the length of the spleen to release the splenocytes into the media. Use a pipette to transfer the free cells and media to a 15 ml centrifuge tube, leaving the spleen casing behind. Allow the tissue debris in the tube to settle for five minutes and transfer the single cell suspension to a fresh tube. Centrifuge the cells for five minutes at 200×g, discard the supernatant and wash the pellet again with cold serum free DMEM. Resuspend the cells in 1 ml serum free DMEM and store the cells on ice until ready for fusion.
Maintain mouse myeloma cells (P3/NS-1/1-Ag4-1 (ATCC accession number TIB-18)) on DMEM with 10% fetal bovine serum. Approximately 10⁷ to 10⁸ cells are required for fusion with B cells obtained from one typical mouse spleen. Immediately prior to hybridoma fusion, harvest myeloma cells from a log phase culture into a 50 ml centrifuge tube and pellet cells by centrifugation at 200×g for five minutes. Remove the supernatant and wash cells twice with serum free DMEM followed by centrifugation at 200×g for five minutes. Resuspend the pellet which should contain 10⁷ to 10⁸ cells in 1 ml serum free DMEM.
Add the spleen cells to the centrifuge tube containing the myeloma cells and centrifuge for five minutes at 500×g. Remove all of the supernatant and loosen the cell pellet by tapping on the side of the tube. Keeping all reagents and cells at 37° C., add 1 ml 50% polyethylene glycol solution (PEG 400, Gibco, Grand Island, N.Y.) dropwise to the tube over a one-minute period with gentle mixing. Allow the mixture to stand at 37° C. for one minute. Add 1 ml warm serum free DMEM dropwise over one minute with gentle mixing. Finally, add 20 ml serum free DMEM dropwise over four minutes, then immediately centrifuge cells at 200×g for five minutes. Discard the supernatant and resuspend cells in 47 ml DMEM containing 20% fetal bovine serum, 0.2 units/ml insulin, 0.5 mM sodium pyruvate, 1 mM oxaloacetic acid, 2 mM L-glutamine, non-essential amino acids, and 10% NCTC-109 lymphocyte media (SDMEM). Add 1 ml volume of cell suspension to the wells of two 24-well flat-bottom tissue culture plates. Include a myeloma cell control well and incubate plates at 37° C. and 10% CO₂.

Following overnight incubation, remove 0.5 ml of media from each well without disturbing the cell layer. Add 1 ml SDMEM containing 0.1 mM hypoxanthine, 0.4 μM aminopterin, and 16 μM thymidine (HAT) to each well and continue incubation at 37° C. and 10% CO₂. Continue to replace 1 ml of used media with 1 ml of fresh DMEM+HAT three times weekly for two to three weeks. When significant clone growth is apparent, assay the wells for the presence of specific antibody by ELISA, indirect FA, or other immunoassay system.

Primary wells testing positive for antibody specific for pathogenic bovine enterovirus should be subcloned immediately to obtain a stable cell line and avoid overgrowth by other clones. Resuspend cells from selected primary wells and perform cell counts using trypan blue stain and a hemocytometer. Make dilutions of the cells to obtain a final concentration of about 2 cells/ml in SDMDM+HAT. Use normal spleen cells obtained from non-incubated mice as a feeder layer by adding 50 μl packed cells per 100 ml media.
Add 200 μl of cell suspension to each well of 96-well plates and incubate at 37° C. and 10% CO₂. Clones should be visible in two to three weeks and supernatants from wells containing single clones are assayed when significant growth is apparent. Repeat the cloning procedure with wells testing positive for specific antibody. Slowly expand selected clones to tissue culture flasks for further characterization and cryopreservation.
Prime BALB/c AnN mice with 0.5 ml pristine given i.p. two weeks before inoculation with hybridoma cells. Harvest hybridoma cells and wash once with Hank's Balanced Salt Solution (HBSS). Resuspend cells in HBSS and inoculate primed mice i.p. with 10⁴ to 10⁶ viable cells. When ascites production is apparent (usually one to two weeks after inoculation) drain by inserting a 16 G 1.5″ needle ventrally in the inguinal region. Hold the hub of the needle over a centrifuge tube and drain ascites into tube. Centrifuge ascite fluid at 200×g, filter through a 0.2 nm filter, and store frozen.

### Diagnostic Assays

The method for diagnosis of pathogenic bovine enterovirus uses polyclonal or monoclonal antibodies. Either the antibody, deep nasal swap sample, the tissue sample, or tissue homogenate can be immobilized by contact with a polystyrene surface or with a surface of another polymer for immobilizing protein. Then the deep nasal swap sample, tissue homogenate, or tissue sample is added (if the antibody is immobilized) or the antibody is added (if the deep nasal swap sample, tissue homogenate or tissue sample is immobilized), incubated and the non-immobilized material is removed, for example, by washing. A labeled species-specific antibody that binds to the polyclonal antibody or monoclonal antibody (depending on which is used) is then added and the presence and quantity of label determined. The label determination indicates the presence of pathogenic bovine enterovirus in the tissue assayed. Typical embodiments of this method include ELISA, RIA, IFA, Northern, Southern, and Western blot immunoassay. Also one can use immuno-PCR techniques, immuno-chemolucent technique, or other detection assays.

While this invention has been described with a reference to specific embodiments, it will be obvious to those of ordinary skill in the art that variations in these methods and compositions may be used and that it is intended that the invention may be practiced otherwise than as specifically described herein. Accordingly this invention includes all modifications encompassed within the spirit and scope of the invention as defined by the claims.

1. An isolated and purified pathogenic bovine enterovirus.

2. The isolated and purified pathogenic bovine enterovirus of claim 1 wherein said pathogenic bovine enterovirus has ATCC deposit number PTA-6306.

3. The isolated and purified pathogenic bovine enterovirus of claim 1 wherein said pathogenic bovine enterovirus is characterized as being insensitive to ether and having a size smaller than 30 nm, said pathogenic bovine enterovirus being specifically reactive with serum antibodies from a bovine, said serum antibodies obtained by intranasally inoculating a bovine with pathogenic bovine enterovirus ATCC deposit number PTA-6306 and collecting serum antibodies from the inoculated bovine 25 to 40 days after inoculation.

4. An immunogenic composition comprising the pathogenic bovine enterovirus of claim 1 wherein said pathogenic bovine enterovirus has been attenuated.

5. An immunogenic composition comprising the pathogenic bovine enterovirus of claim 1 wherein said pathogenic bovine enterovirus has been inactivated.

6. An immunogenic composition comprising the pathogenic bovine enterovirus of claim 1 wherein said pathogenic bovine enterovirus has ATCC deposit number PTA-6306 and wherein said pathogenic bovine enterovirus has been attenuated or inactivated.

7. An immunogenic composition for generating an immune response in a bovine comprising:
an inactivated virus which is the causative agent for pathogenic bovine enterovirus disease, said virus characterized as being insensitive to ether and having a size smaller than 30 nm, said virus being specifically reactive with serum antibodies from a bovine, said serum antibodies obtained by intranasally inoculating a bovine with pathogenic bovine enterovirus ATCC deposit number PTA-6306 and collecting serum antibodies from the inoculated bovine 25 to 40 days after inoculation.

8. An immunogenic composition for generating an immune response in a bovine comprising:
an attenuated virus which is the causative agent for pathogenic bovine enterovirus disease, said virus characterized as being insensitive to ether and having a size smaller than 30 nm, said virus being specifically reactive with serum antibodies from a bovine, said serum antibodies obtained by intranasally inoculating a bovine with pathogenic bovine enterovirus ATCC deposit number PTA-6306 and collecting serum antibodies from the inoculated bovine 25 to 40 days after inoculation.

9. A method for producing the immunogenic composition of claim 4 comprising:
homogenizing tissue from bovine affected with pathogenic bovine enterovirus disease to form a homogenate which includes pathogenic bovine enterovirus; and
attenuating the pathogenic bovine enterovirus in the homogenate by a process comprising passaging the pathogenic bovine enterovirus through animal cells to form attenuated pathogenic bovine enterovirus which is non-zoopathogenic in bovine.

10. A method for producing the immunogenic composition of claim 4 comprising:
passaging the pathogenic bovine enterovirus through animal cells to form attenuated pathogenic bovine enterovirus which is non-zoopathogenic in bovine.

11. A method for producing the immunogenic composition of claim 5 comprising:
homogenizing tissue from bovine affected with pathogenic bovine enterovirus disease to form a homogenate which includes pathogenic bovine enterovirus;
inoculating a cell culture with the homogenate to grow up pathogenic bovine enterovirus;
harvesting pathogenic bovine enterovirus from the cell culture; and
inactivating the pathogenic bovine enterovirus by adding an inactivating agent.

12. A method for producing the immunogenic composition of claim 5 comprising:
inactivating the pathogenic bovine enterovirus by adding an inactivating agent.

13. A method for diagnosing pathogenic bovine enterovirus disease in bovine comprising:
obtaining a lung tissue sample from the bovine;
forming a liquid homogenate of the sample;
immobilizing the liquid homogenate on a support;
adding a monoclonal antibody to the immobilized homogenate to form a complex with a viral agent in the liquid homogenate, wherein the monoclonal antibody specifically binds to a protein of pathogenic bovine enterovirus ATCC deposit number PTA-6306; and detecting the complex, wherein the presence of the complex is indicative of the presence of pathogenic bovine enterovirus disease.

14. A method for diagnosing pathogenic bovine enterovirus disease in bovine comprising:
forming a liquid homogenate of a tissue sample from the bovine suspected of being infected with pathogenic bovine enterovirus;
adding a monoclonal antibody to the liquid homogenate to form a precipitated complex with a viral agent in the homogenate, wherein the monoclonal antibody specifically binds to a protein of pathogenic bovine enterovirus ATCC deposit number PTA-6306; and detecting the precipitated complex, wherein the presence of the precipitated complex is diagnostic of pathogenic bovine enterovirus disease.

15. A method for diagnosing pathogenic bovine enterovirus disease in bovine comprising:
obtaining a lung tissue sample from the bovine;
forming a liquid homogenate of the tissue sample;
incubating the liquid homogenate with a cell preparation under conditions effective to culture the cells;
processing the cultured cells to provide a cell scrapping;
immobilizing the cell scraping on a support;
adding a monoclonal antibody to the immobilized cells scrapping to form a complex with a viral agent in the immobilized cells scrapping, wherein the monoclonal antibody specifically binds to a protein of pathogenic bovine enterovirus ATCC deposit number PTA-6306; and
detecting the complex, wherein the presence of the complex is diagnostic of pathogenic bovine enterovirus disease.

16. A method for diagnosing pathogenic bovine enterovirus comprising:
obtaining a lung tissue sample from a bovine;
immobilizing the lung tissue sample on a support;
adding a monoclonal antibody to the immobilized lung tissue sample to form a complex with a viral agent in the immobilized lung tissue sample, wherein the monoclonal antibody specifically binds to a protein of pathogenic bovine enterovirus ATCC deposit number PTA-6306; and
detecting the complex, wherein the presence of the complex is diagnostic of pathogenic bovine enterovirus disease.

17. A method of generating an immune response in a bovine against pathogenic bovine enterovirus comprising administering the immunogenic composition of claim 4 to a bovine.

18. A monoclonal antibody that binds to a protein of pathogenic bovine enterovirus ATCC deposit number PTA-6306.
